(19) United States
(12) Patent Application Publication
(10) Pub. No.: US 2025/0069695 A1
(43) Pub. Date: Feb. 27, 2025

(54) METHOD AND SYSTEM FOR SORTING DRUG-RESPONSIVE CELL POPULATION BASED ON SINGLE-CELL RNA SEQUENCING (scRNA-seq) DATA

(71) Applicant: Innovation Center of Yangtze River Delta, ZJU, Jiaxing City (CN)

(72) Inventors: Xiaohui FAN, Jiaxing City (CN); Xin SHAO, Jiaxing City (CN); Chengyu LI, Jiaxing City (CN); Jie LIAO, Jiaxing City (CN)

(73) Assignee: Innovation Center of Yangtze River Delta, ZJU, Jiaxing City (CN)

(21) Appl. No.: 18/440,423

(22) Filed: Feb. 13, 2024

(30) Foreign Application Priority Data
Aug. 21, 2023 (CN) 202311052732.0

(51) Int. Cl. G16B 30/00

(52) U.S. Cl. CPC G16B 30/00

(57) ABSTRACT

Method and systems for sorting drug-responsive cell population based on single-cell RNA sequencing (scRNA-seq) data are disclosed. In some examples, the method includes: constructing a target gene regulatory network (GRN) for each of the cell populations based on scRNA-seq data of cell populations in a disease state; virtually knocking out targets in the GRN, and constructing a target-perturbed gene regulatory network (tpGRN); acquiring a low-dimensional representation of each of network nodes from the GRN in the GRN and the tpGRN through manifold alignment; calculating a Euclidean distance for each of the network nodes between the GRN and the tpGRN through a Euclidean distance; scoring a drug response of each of the cell populations by comprehensively considering changing trends of a drug target, a 2-hop node, and an edge of the 2-hop node in the GRN and the tpGRN, and determining a sorting result of the drug-responsive cell population.

### CROSS REFERENCE TO RELATED APPLICATIONS

This application claims priority to Chinese patent application number 202311052732.0, filed on Aug. 21, 2023, the disclosure of which is incorporated by reference herein in its entirety.

### FIELD OF THE DISCLOSURE

The disclosure relates generally to the field of sorting of drug-responsive cell populations. More specifically, the disclosure relates to methods and systems for sorting a drug-responsive cell population based on single-cell RNA sequencing (scRNA-seq) data.

### BACKGROUND

Single-cell RNA sequencing (scRNA-seq) allows measurement of all gene expressions in each single cell and has been widely used in research of cell heterogeneity. In consideration of the high heterogeneity of cellular responses to drugs, it is crucial to determining the cell populations most sensitive to drug perturbations using scRNA-seq data so as to understand the drug mechanisms of action and guiding the drug development, which has received widespread attention. Currently, the most common approach is to score and sort drug-responsive cell populations using the number of differentially expressed genes (DEGs) between paired cell populations under different conditions. Recently, some researchers have also proposed a method called Augur to conduct perturbation scoring and sorting in cell populations. This method prioritizes cell types that are most sensitive to biological perturbations by exploiting the separability of a same cell population under different conditions in a high-dimensional space. However, drugs generally rely on binding to target proteins to exert their therapeutic effects. Obviously, these methods mentioned above do not take into account the prior knowledge of drug targets, resulting in inaccurate inference of drug-responsive cell populations. In addition, existing strategies and methods require scRNA-seq data in two different states, severely limiting their applications in the scRNA-seq data of only disease states. As of now, there is no method to predict drug-responsive cell populations using only scRNA-seq data in disease states.

### SUMMARY

The following presents a simplified summary of the invention in order to provide a basic understanding of some aspects of the invention. This summary is not an extensive overview of the invention. It is not intended to identify critical elements or to delineate the scope of the invention. Its sole purpose is to present some concepts of the invention in a simplified form as a prelude to the more detailed description that is presented elsewhere.

In some embodiments, the disclosure provides a method for sorting a drug-responsive cell population based on scRNA-seq data, including the following steps.

Acquiring scRNA-seq data and drug target information of cell populations in a disease state.

Constructing a target gene regulatory network (GRN) for each of the cell populations based on a co-expression relationship of genes and the scRNA-seq data.

Virtually knocking out targets in the GRN according to the drug target information, and constructing a target-perturbed gene regulatory network (tpGRN) for each of the cell populations.

Acquiring a low-dimensional representation of each of network nodes from the GRN in the GRN and the tpGRN through manifold alignment. The network nodes are gene nodes.

Calculating a Euclidean distance for each of the network nodes between the GRN and the tpGRN through a Euclidean distance based on the low-dimensional representation.

Scoring a drug response of each of the cell populations based on the distance by comprehensively considering changing trends of a drug target, a 2-hop node, and an edge of the 2-hop node in the GRN and the tpGRN, and generating a drug perturbation score.

Sorting the drug response of each of the cell populations according to the drug perturbation score, and determining a sorting result of the drug-responsive cell population. The sorting result of the drug-responsive cell population is used to characterize a degree of response of each of the cell populations to drug perturbation.

Optionally, a process of constructing the GRN for each of the cell populations based on the co-expression relationship of the genes and the scRNA-seq data includes following steps.

Constructing a scRNA-seq expression matrix according to the scRNA-seq data.

Randomly selecting δc cells from each of cell types in the scRNA-seq expression matrix, extracting a gene expression matrix corresponding to each of the selected δc cells, and repeating the above operations S times. δ represents a selection ratio and c represents a number of cells.

Subjecting genes in the gene expression matrix to feature selection to construct a gene set of the GRN. The gene set includes top 2,000 highly variable genes in the scRNA-seq data, genes corresponding to transcription factors in an AnimalTFDB database, and target genes of drugs in a DGIdb database.

Determining a low-dimensional representation of each of the genes in the gene set by principal component analysis and estimating a regression coefficient between the genes as a weight of an edge in the GRN.

Defining any one of the gene expression matrices, subjecting a target gene on multiple potential covariations using the principal component analysis, and constructing a relationship between the target gene and a regulatory gene.

Constructing a gene adjacency matrix according to the relationship between the target gene and the regulatory gene as well as the weight.

Combining multiple gene adjacency matrices into a third-order tensor χ of S×T×R using tensor component analysis. S represents a number of the GRNs, T represents a number of the target genes, and R represents a number of the regulatory genes.

Extracting a main feature pattern shared by a plurality of the GRNs, decomposing and approximating the third-order tensor χ to generate a new third-order tensor.

Conducting normalization on a result obtained through dividing each of the weights by a maximum absolute value in all weight absolute values based on the new third-order tensor, and constructing a final adjacency matrix for each of the cell populations. The final adjacency matrix is the GRN.

Optionally, the relationship between the target gene and the regulatory gene is:

$$Y = {X}^{′} {β}^{′} + ε .$$

Y represents an expression vector of the target gene, X′ represents expression vectors of the regulatory genes corresponding to multiple top-ranked principal components, β′ represents a regression coefficient of a transformed potential covariate, and ε represents a random error.

Optionally, a process of extracting the main feature pattern shared by the multiple GRNs, decomposing and approximating the third-order tensor χ to generate the new third-order tensor includes following steps.

Extracting the main feature pattern shared by the multiple GRNs, and decomposing and approximating the third-order tensor χ with a formula χ≈Σᵣ₌₁^{R}=s^{r}⊗t^{r}⊗r^{r}, Here, s^{r} represents a vector of a factor r in a GRN number dimension, ⊗ represents an outer product operation, t^{r} represents a vector of the factor r in a target gene dimension, and r^{r} represents a vector of the factor r in a regulatory gene dimension.

Selecting first five factors to characterize key components of the third-order tensor χ, and generating a new third-order tensor through cumulative averaging of the key components.

Optionally, a process of virtually knocking out the targets in the GRN according to the drug target information, and constructing the tpGRN for each of the cell populations includes following steps.

Extracting a row where a drug target gene is located from the final adjacency matrix, and setting a corresponding value of the row where the drug target gene is located to 0, to virtually construct a drug-perturbed adjacency matrix. The drug-perturbed adjacency matrix is the tpGRN.

Optionally, a process of acquiring the low-dimensional representation of each of the network nodes from the GRN in the GRN and the tpGRN through manifold alignment includes following steps.

Combining the final adjacency matrix and the drug-perturbed adjacency matrix into a combined matrix.

Projecting the combined matrix to a shared low-dimensional potential space with the manifold alignment, determining the low-dimensional representation of each of the gene nodes in the GRN and the tpGRN, and interpreting the Euclidean distance between each of matched gene nodes as a perturbation effect of a drug on the cell types.

Optionally, the combined matrix is:

$$W = [ \begin{matrix}{A}^{′} & λ I \\ λ I & {A}_{PB}^{′}\end{matrix} ] .$$

W represents the combined matrix, A′ represents the final adjacency matrix, A_{PB}′ represents the drug-perturbed adjacency matrix, λ represents an adjustment parameter, and/represents an identity matrix describing a correspondence between the gene nodes in the GRN and the tpGRN.

Optionally, the drug perturbation score score is:

$$score = \frac{{D}_{Tar} {W}_{o u t}}{{Deg}_{T a r}} + {\sum }_{n}^{N} {D}_{n} {W}_{i n} + {\sum }_{n}^{N} \frac{{D}_{n} {W}_{o u t}}{{Deg}_{n}} .$$

D_{Tar} represents the distance of the target gene between the GRN and the tpGRN, Wₒᵤₜ represent a weight of an edge of a gene node leaving the GRN, Deg_{Tar} is a degree of the target gene in the GRN, N represents a total number of genes regulated by the target gene, n represents a gene sequence number, Dₙ represents a distance of a downstream gene between the GRN and the tpGRN, Wᵢₙ represents a weight of an edge of a gene node entering the GRN, and Degₙ represents a degree of the downstream genes in the GRN.

The present disclosure further provides a system for sorting a drug-responsive cell population based on scRNA-seq data, including following items.

A data and information acquisition module configured to acquire scRNA-seq data and drug target information of cell populations in a disease state.

A GRN construction module configured to construct a target gene regulatory network (GRN) for each of the cell populations based on a co-expression relationship of genes and the scRNA-seq data.

A tpGRN construction module configured to virtually knock out targets in the GRN according to the drug target information, and construct a target-perturbed gene regulatory network (tpGRN) for each of the cell populations.

A manifold alignment configured to acquire a low-dimensional representation of each of network nodes from the GRN in the GRN and the tpGRN through manifold alignment. The network node is a gene node.

A Euclidean distance calculation module configured to calculate a Euclidean distance for each of the network nodes in the GRN and the tpGRN through a Euclidean distance based on the low-dimensional representation.

A drug perturbation score generation module configured to score a drug response of each of the cell populations based on the distance by comprehensively considering changing trends of a drug target, a 2-hop node, and an edge of the 2-hop node in the GRN and the tpGRN, and generate a drug perturbation score.

A sorting module configured to sort the drug response of each of the cell populations according to the drug perturbation score, and determine a sorting result of the drug-responsive cell population. The sorting result of the drug-responsive cell population is used to characterize a degree of response of each of the cell populations to drug perturbation.

### BRIEF DESCRIPTION OF THE DRAWINGS

Illustrative embodiments of the present disclosure are described in detail below with reference to the attached drawing figures. The accompanying drawings in the following description show merely some embodiments of the present disclosure, and other drawings may be derived from these accompanying drawings by those of ordinary skill in the art without creative efforts.

FIG. 1 shows a flow chart of the method for sorting a drug-responsive cell population based on scRNA-seq data according to an embodiment of the disclosure.

FIG. 2 shows an scRank working architecture diagram according to an embodiment of the disclosure.

FIG. 3A shows a schematic diagram of SERGIO generating the simulated data of a performance test for the scRank on simulated data according to an embodiment of the disclosure.

FIG. 3B shows a schematic diagram of application scenario 1 according to an embodiment of the disclosure, which generates simulated data where an activity of a same gene co-expression module decreases in an orderly manner.

FIG. 3C shows a schematic diagram of application scenario 2 according to an embodiment of the disclosure, which generates simulated data where the activity of the gene co-expression module has a significant difference.

FIG. 3D shows a schematic diagram of application scenario 3 according to an embodiment of the disclosure, which generates simulated data where the activity of the gene co-expression module has cell type-specificity according to an embodiment of the disclosure.

FIG. 4A shows a schematic diagram of a prediction result of the scRank on mouse medulloblastoma according to an embodiment of the disclosure.

FIG. 4B shows a schematic diagram of cell populations confirmed to be sensitive and resistant to vismodegib in the literature according to an embodiment of the disclosure.

FIG. 4C shows a schematic diagram of a GRN of Node_A and Node_B under disease conditions according to an embodiment of the disclosure.

FIG. 4D shows a schematic diagram of GRNs in two tumor subtypes and their differences before and after administration according to an embodiment of the disclosure.

FIG. 4E shows a schematic diagram of a GO enrichment result of gene modules M1 and M2 according to an embodiment of the disclosure.

FIG. 4F shows a schematic diagram of DEGs in the Node_A and Node_B under disease state according to an embodiment of the disclosure.

FIG. 4G shows a schematic diagram of a significantly activated pathway in the Node_B and Node_A according to an embodiment of the disclosure.

FIG. 4H shows a prognostic analysis chart of medulloblastoma patients using feature genes of the Node_A and Node_B as classification criteria according to an embodiment of the disclosure.

FIG. 5A shows a schematic diagram of the scRNA-seq data of patients with major depressive disorder (MDD) and a target SLC6A4 of a therapeutic drug fluoxetine according to an embodiment of the disclosure.

FIG. 5B shows a schematic diagram of ranking results of all cell populations by the scRank on a data set of the patients with MDD according to an embodiment of the disclosure.

FIG. 5C shows a schematic diagram of the GRN and cluster analysis results of Ex_9 and Inhib_5 in disease states according to an embodiment of the disclosure.

FIG. 5D shows a schematic diagram of pathway enrichment results of gene co-expression modules M1, M2, M3, and M4 according to an embodiment of the disclosure.

FIG. 5E shows a schematic diagram of activity distribution of the gene co-expression module M2 for all cell populations in MDD according to an embodiment of the disclosure.

FIG. 5F shows a schematic diagram of differential genes of Ex_9 in normal person and MDD according to an embodiment of the disclosure.

FIG. 5G shows a schematic diagram of pathways significantly enriched in Ex_9

FIG. 5H shows spatial distribution of different cell populations in MDD on a mouse forebrain according to an embodiment of the disclosure.

FIG. 5I shows spatial distribution of different cell populations in MDD on a human dorsolateral prefrontal cortex according to an embodiment of the disclosure.

FIG. 6A shows a schematic diagram comparing the performances of scRank, Augur, and Wilcox with simulated data according to an embodiment of the disclosure.

FIG. 6B shows a schematic diagram of five sets of real drug-perturbed scRNA-seq data sets according to an embodiment of the disclosure.

FIG. 6C shows a schematic diagram comparing the performances of scRank, Augur, Wilcox, MAST, bimod, and DESeq2 with simulated data according to an embodiment of the disclosure.

FIG. 6D shows box distribution of the prediction results with different methods according to an embodiment of the disclosure.

### DETAILED DESCRIPTION

The following describes some non-limiting exemplary embodiments of the invention with reference to the accompanying drawings. The described embodiments are merely a part rather than all of the embodiments of the invention. All other embodiments obtained by a person of ordinary skill in the art based on the embodiments of the disclosure shall fall within the scope of the disclosure.

### Example 1

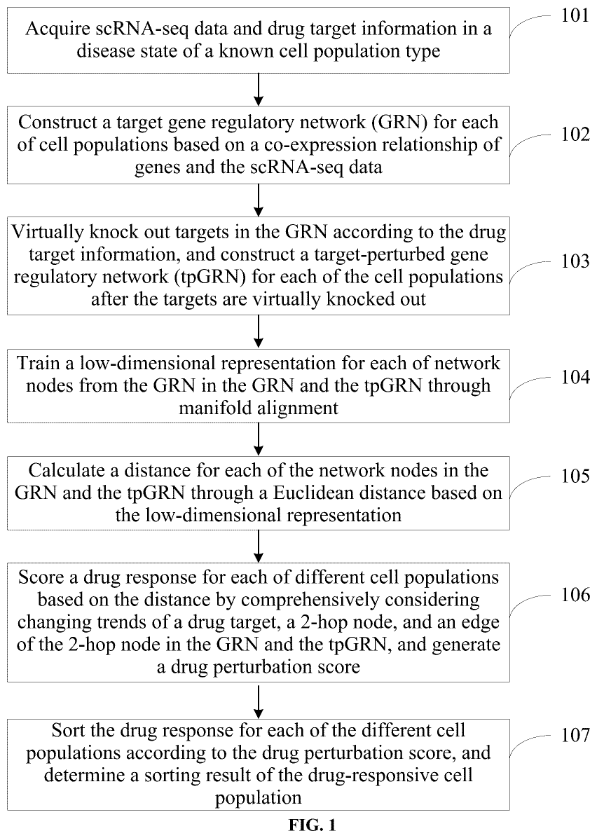

As shown in FIG. 1, the present disclosure provided a method for sorting a drug-responsive cell population based on scRNA-seq data, including the following steps:
Step 101: scRNA-seq data and drug target information of cell populations in a disease state were acquired.
Step 102: a target gene regulatory network (GRN) was constructed for each of the cell populations based on a co-expression relationship of genes and the scRNA-seq data.

In practical applications, step 102 specifically included: constructing a scRNA-seq expression matrix according to the scRNA-seq data; randomly selecting δc cells from each of cell types in the scRNA-seq expression matrix, extracting a gene expression matrix corresponding to each of the selected δc cells, and repeating the above operations S times to obtain S GRNs; where δ represented a selection ratio and c represented a number of cells; subjecting genes in the gene expression matrix to feature selection to construct a gene set of the GRN; where the gene set may include top 2,000 highly variable genes in the scRNA-seq data, genes corresponding to transcription factors in an AnimalTFDB database, and target genes of drugs in a DGIdb database; determining a low-dimensional representation of each of the genes in the gene set by principal component analysis and estimating a regression coefficient between the genes as a weight of an edge in the GRN; defining any one of the gene expression matrices, subjecting a target gene on multiple potential covariations using the principal component analysis, and constructing a relationship between the target gene and a regulatory gene; constructing a gene adjacency matrix according to the relationship between the target gene and the regulatory gene as well as the weight; combining multiple gene adjacency matrices into a third-order tensor χ of S×T×R using tensor component analysis; where S represented a number of the GRNs, T represented a number of the target genes, and R represented a number of the regulatory genes; extracting a main feature pattern shared by a plurality of the GRNs, decomposing and approximating the third-order tensor χ to generate a new third-order tensor; and conducting normalization on a result obtained through dividing each of the weights by a maximum absolute value in all weight absolute values based on the new third-order tensor, and constructing a final adjacency matrix for each of the cell populations; where the final adjacency matrix is the GRN.

As an optional implementation manner of the present disclosure, a construction process of the GRN included:

For a given cell population i (i∈{1, 2, . . . , C}, the scRNA-seq expression matrix was Mᵢ[n×c] (including n genes and c cells), C represented a total number of cell types in the data set. Taking into account the heterogeneity between cell populations, the method for ranking a drug-responsive cell population based on scRNA-seq data (scRank): δc cells were randomly selected from Mᵢ for t times, and a set of gene expression matrix 0=[X₁, . . . , Xₜ] with a number of δc cells was simulated, and then used to construct a cell group-specific GRN. δ represented a selection ratio, δ∈(0,1).

In order to retain meaningful biological data while reducing computational costs, feature selection was conducted on the genes of sequencing data using the scRank. These feature genes mainly included: (1) top 2,000 highly variable genes (HVGs) in the scRNA-seq data; (2) genes corresponding to transcriptional factors (TFs) in an AnimalTFDB (guolab.wchscu.cn/AnimalTFDB #!/) database; and (3) target genes of drugs or inhibitors in a DGIdb (dgidb.genome.wustl.edu) database. In addition, the scRank removed mitochondria, ribosomes, and other redundant genes to retain differences between cells and meaningful biological processes. Therefore, a final gene set used to construct GRN was G=[HVG+TF+Tar], and a number was represented by p.

Based on the predefined cells and genes, the scRank adopted principal component analysis (PCA) to obtain a low-dimensional representation of each gene, and then estimate a regression coefficient between genes as a weight of the edge in the GRN. Given a gene expression matrix X[n×p], Y_{n×1}=(y₁, . . . , yₙ)^{T} represents the expression vector of the target gene, and X_{n×(p-1)}=(x₁, . . . xₙ)^{T} represents the expression vector of the remaining p-1 regulatory genes. Assuming that Y depends on X, the relationship between the target gene and other genes could be modeled by the following formula:

$$Y = X β + ε . \qquad ( 1 )$$

β∈ [symbol] ^{p-1} represented a regression coefficient vector under the p−1 dimension in the real number space, and ε represented the random error. To reduce computational cost and overfitting, scRank utilized the PCA to regress target genes on k potential covariations.

Assuming that X_{n×k}′=X_{n×(p-1)}V_{(p-1)×k} represented the selected top-ranked k principal components and X_{n×(p-1)} was the original data, the regression problem could be transformed into:

$$Y = {X}^{′} {β}^{′} + ε . \qquad ( 2 )$$

In the above formula (2), V_{(p-1)×k} was obtained from X through singular value decomposition, and represented a load matrix with p−1 rows and k columns, where each column represented the load of a principal component. β′∈ [symbol] ^{k} represented the transformed regression coefficient, which was estimated by an ordinary least squares method.

The correlation between the target gene and other genes was calculated by the following formula:

$$\hat{β} = {V}_{k} {\hat{β}}^{′} ∈ {ℝ}^{p - 1} . \qquad ( 3 )$$

In the above formula (3), \hat{β} and \hat{β}′ represent the parameter estimates of the original regression coefficient β and the transformed regression coefficient β′, respectively.

By using the first k principal component loadings V as weights, the regression coefficient β′ of the transformed potential covariate could be converted into the regression coefficient β of the original variable; the regression through dimensionality reduction transformation could alleviate multicollinearity problems and increase computational efficiency.

For each randomly selected iteratively regressed feature gene in G, t adjacency matrices containing the relationship and strength of feature gene pairs in cell type i.

In order to retain important biological signals in GRN, the scRank only retained the top 5% absolute weight values and removed autocorrelated coefficients, and the weight values were used to construct the final gene adjacency matrix A.

In order to improve the robustness of GRN and maintain heterogeneity, the scRank extracted multiple GRNs from randomly selected data and used tensor component analysis (TCA) to integrate them into a matrix. Specifically, t GRNs were combined into a third-order tensor χ of S×T×R, where S, T, and R corresponded to the number of GRNs, the number of target genes, and the number of regulatory genes, respectively. In order to reduce the noise of the gene adjacency matrix A, the main feature patterns shared among multiple GRNs were extracted, and χ were decomposed and approximated through CANDECOMP/PARAFAC, a formula was as follows:

$$χ \approx {\sum }_{r = 1}^{R} {s}^{r} ⊗ {t}^{r} ⊗ {r}^{r} . \qquad ( 4 )$$

⊗ represented the outer product, and s^{r}, t^{r}, and r^{r} represented vectors of factors r, containing the loadings of each element in each dimension of the tensor. In practical applications, the scRank selected the first five factors to represent the key components of χ, and obtained a new tensor χ′ through cumulative averaging of the key components; and each weight was divided by a maximum absolute value of the tensor for normalization, to obtain the final GRN for a given cell type i, denoted as A′.

Step 103: targets in the GRN were virtually knocked out according to the drug target information, and a target-perturbed gene regulatory network (tpGRN) for each of the cell populations was constructed.

In practical applications, step 103 specifically included: extracting a row where a drug target gene is located from the final adjacency matrix, and setting a corresponding value of the row where the drug target gene is located to 0, to virtually construct a drug-perturbed adjacency matrix; where the drug-perturbed adjacency matrix is the tpGRN.

Step 104: a low-dimensional representation of each of network nodes from the GRN in the GRN and the tpGRN was acquired through manifold alignment; where the network nodes were gene nodes.

In practical applications, step 104 specifically included: combining the final adjacency matrix and the drug-perturbed adjacency matrix into a combined matrix; and projecting the combined matrix to a shared low-dimensional potential space with the manifold alignment, determining the low-dimensional representation of each of the gene nodes in the GRN and the tpGRN, and interpreting the Euclidean distance between each of matched gene nodes as a perturbation effect of a drug on the cell types.

As an optional implementation of the present disclosure, GRN virtual knockout and manifold alignment included the following steps:

In order to simulate the impact of drugs or inhibitors on GRN, the scRank extracted the row of the drug target gene from the adjacency matrix A′ and set all values in it to 0, thereby virtually constructing the drug-perturbed adjacency matrix A_{PB}′, namely the tpGRN. Manifold alignment was conducted to jointly project the two high-dimensional adjacency matrices of the original GRN and tpGRN into a shared low-dimensional potential space, and the distance between each matching gene node was interpreted as a perturbation effect of the drug on the target cell type.

The manifold alignment projected two data sets generated by a similar process into a new low-dimensional space that not only maintained a minimum distance between matching points but also retained an original structure. Therefore, the larger the distance between gene nodes was, the greater the change in the topological structure of the node was in the two data sets. The scRank achieved manifold alignment by combining A′∈ [symbol] ^{p×p} and A_{PB}′∈ [symbol] ^{p×p} into a combined matrix W. The specific formula was as follows:

$$W = [ \begin{matrix}{A}^{′} & λ I \\ λ I & {A}_{PB}^{′}\end{matrix} ] . \qquad ( 5 )$$

The variable names in rows and columns in A′ and A_{PB}′ were the gene set G used to construct the GRN, I represented the identity matrix describing the correspondence between the gene nodes in the two GRNs, and λ was the adjustment parameter.

Based on the shared low-dimensional projection, the scRank solved d minimum non-zero eigenvalues using a Laplacian eigenmap problem. E_{2p×d}=(e₁, . . . , e₂ₚ) was let represent the feature vector, and the Euclidean distance between two gene nodes was calculated through D(p)=∥eₚ−e₂ₚ∥=(d₁, . . . , dₚ), where e₂ₚ was the e₂ₚ feature vector. The Euclidean distance here reflected the impact of target perturbation on gene nodes and simulated the effect of drug action and gene network.

Step 105: a distance for each of the network nodes between the GRN and the tpGRN was calculated through a Euclidean distance based on the low-dimensional representation.

Step 106: a drug response of each of the cell populations was scored based on the distance by comprehensively considering changing trends of a drug target, a 2-hop node, and an edge of the 2-hop node in the GRN and the tpGRN, and a drug perturbation score was generated.

Step 107: the drug response of each of the cell populations was sorted according to the drug perturbation score, and a sorting result of the drug-responsive cell population was determined; where the sorting result of the drug-responsive cell population was used to characterize a degree of response of each of the cell populations to drug perturbation.

As an optional embodiment of the present disclosure, a process of sorting the drug-responsive cell population included the following steps:

In order to more accurately infer the degree of response of cell populations to drugs, the scRank introduced a diffusion effect of the network. That is, a target gene Tar was given, the impact of drug perturbation might propagate from the target gene to the downstream genes of the target gene 2-hop; and this impact was positively related to the weight of the edge and the distance between nodes. A specific calculation formula was as follows:

$$score = \frac{{D}_{Tar} {W}_{o u t}}{{Deg}_{T a r}} + {\sum }_{n}^{N} {D}_{n} {W}_{i n} + {\sum }_{n}^{N} \frac{{D}_{n} {W}_{o u t}}{{Deg}_{n}} . \qquad ( 6 )$$

D_{Tar} and Dₙ were obtained through formulas (1) to (5) and represented the distance of the target gene and downstream genes between the GRN and tpGRN before and after virtual knockout; and Deg represented a degree of the corresponding gene node in the GRN network, while Wᵢₙ and Wₒᵤₜ represented the weights of the edges entering and leaving the node in GRN, respectively. Therefore, in the formulas (1) to (6), the first term represented the normalized drug perturbation effect of the target gene; the second term represented the normalized diffusion effect of the 1-hop node of the target gene; and the third term represented the normalized diffusion effect of the 2-hop node of the target gene. By combining a direct perturbation of the drug on the gene node in step (3) and a diffusion effect of the perturbation in the network, a comprehensive drug perturbation score was calculated as “score”. The drug response of the cell population was scored and ranked according to the score.

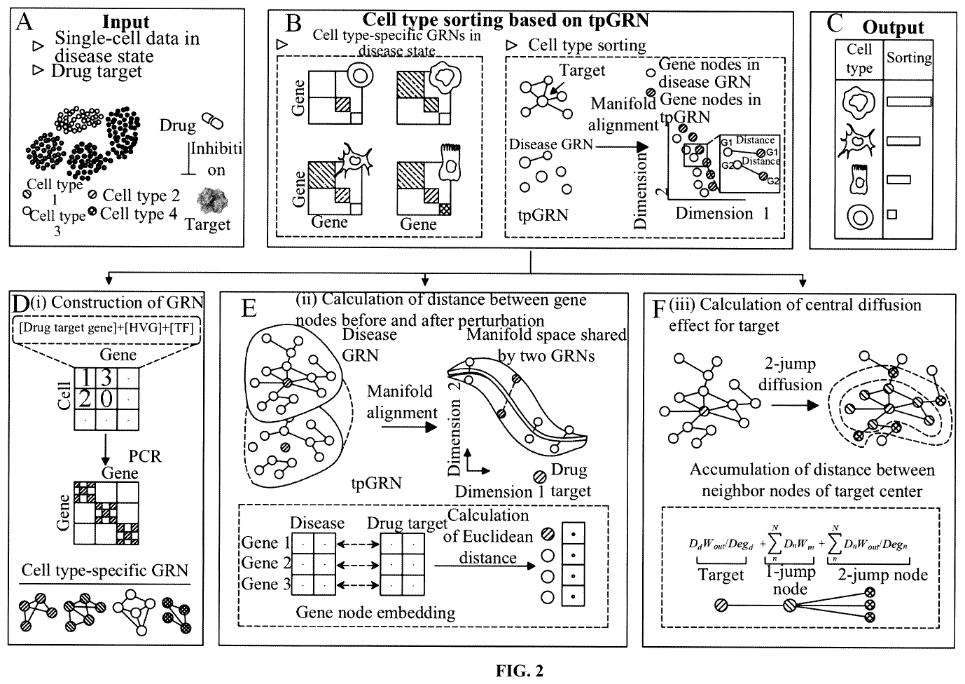

In the present disclosure, the method (scRank) for sorting a drug-responsive cell population based on scRNA-seq data was mainly divided into three layers: a data input layer of portion A in FIG. 2, a cell type sorting layer of portion B in FIG. 2, and a data output layer of portion C in FIG. 2; and the cell type sorting layer was further divided into portion D in FIG. 2, portion E in FIG. 2, and portion F in FIG. 2.

More specifically: portion A in FIG. 2 shows a schematic diagram of a data input layer of a scRank working architecture diagram according to an embodiment of the disclosure; portion B in FIG. 2 shows a schematic diagram of a cell type sorting layer of the scRank working architecture diagram according to an embodiment of the disclosure; portion C in FIG. 2 shows a schematic diagram of a data output layer of the scRank working architecture diagram according to an embodiment of the disclosure; portion D in FIG. 2 shows a schematic diagram of constructing the GRN of the scRank working architecture diagram according to an embodiment of the disclosure; portion E in FIG. 2 shows a schematic diagram of calculating a distance of the gene node between the GRN and the tpGRN before and after perturbation of the scRank working architecture diagram according to an embodiment of the disclosure; and portion F in FIG. 2 shows a schematic diagram of calculating a central diffusion effect of the target of the scRank working architecture diagram according to an embodiment of the disclosure.

An input of the scRank was the scRNA-seq data and drug target information in a disease state of a known cell type. The cell population ranking algorithm of scRank mainly included three parts. A first part was to construct the GRN of each cell population based on a co-expression relationship of genes. A second part was to virtually knock out targets in GRN based on the drug target information, and construct tpGRN after virtual knockout for each cell population; and to train the low-dimensional representation of each network node in GRN before and after virtual knockout through manifold alignment. A third part was to calculate the distance of each network node between the GRN and the tpGRN before and after virtual knockout through Euclidean distance, and score the drug responses of the cell populations by comprehensively considering changing trends of drug targets as well as 2-hop nodes and edges thereof in the GRN before and after virtual knockout. An output of the scRank was a result of rank sum ratio, which represented a degree of response of the cell population to drug perturbation.

### Example 2

In order to conduct the method corresponding to Example 1, scRNA-seq simulated data was generated through a Single-cell Expression of Genes In silico (SERGIO) and a predefined GRN module. According to the scientific hypothesis that the effect of drug perturbation depended on gene activity, drug-responsive and non-drug-responsive cell populations were defined, that is, positive and negative samples. Considering that perturbation effects dominated in densely connected subnetworks compared to sparsely connected subnetworks. Therefore, three application scenarios were designed using simulation-generated data to benchmark the scRank, to evaluate a predictive performance of the scRank in the task of identifying drug-responsive cell populations.

### (1) Construction of Simulated Data:

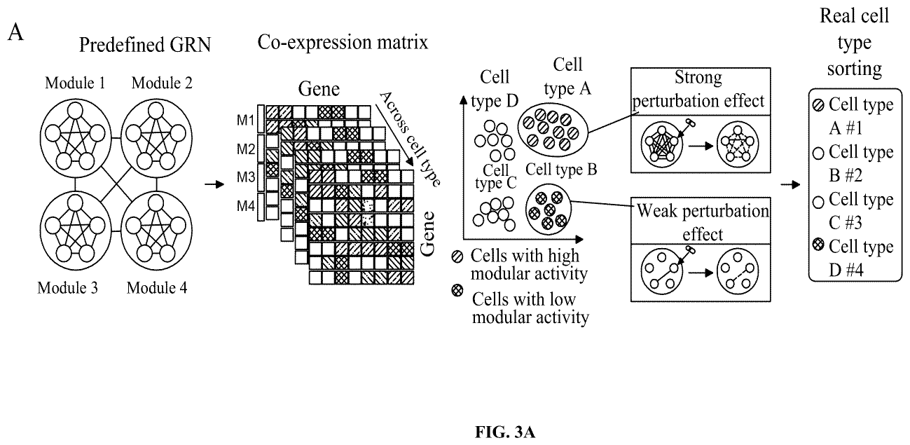

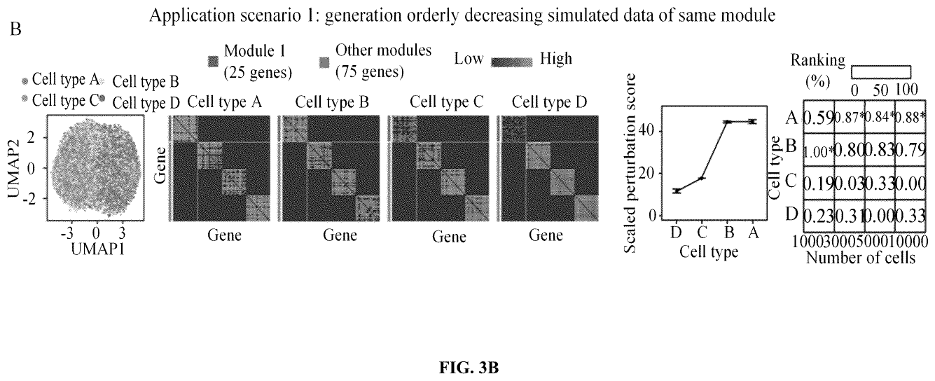

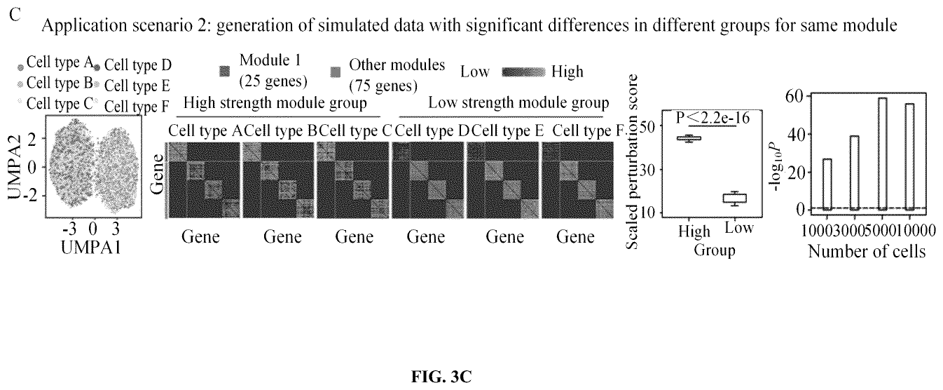

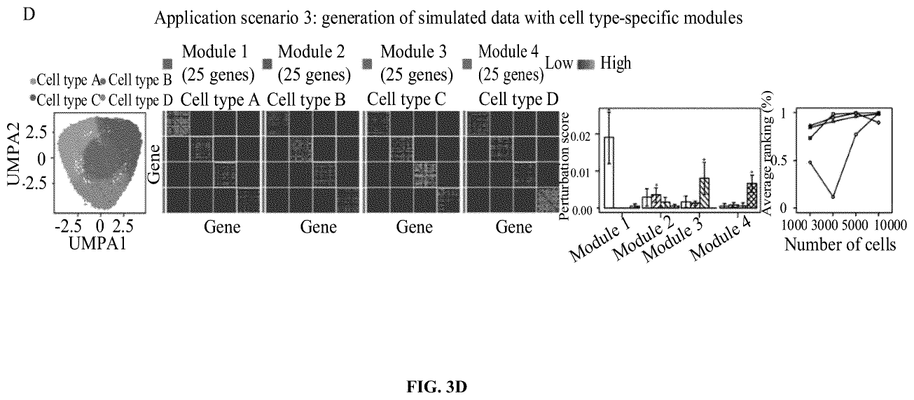

In order to construct simulated data consistent with real application scenarios, a gene expression profile matrix of a specific cell population was automatically generated using a Python package of the “SERGIO” and the predefined GRN module. GRN contained four gene co-expression modules, each of which contained 25 genes. A gene co-expression intensity within each module was adjusted according to the two parameters of adjusting productivity and interaction strength to simulate an expression activity of specific gene modules of the cell population. By adjustment parameter and GRN, three different scRNA-seq simulated data and their corresponding three application scenarios were designed to benchmark the scRank, as shown in FIGS. 3A-D. FIGS. 3A-D showed a schematic diagram of a performance test for the scRank on simulated data; where FIG. 3A was a schematic diagram of SERGIO generating the simulated data; FIG. 3B was a schematic diagram of application scenario 1, which generated simulated data where an activity of a same gene co-expression module decreased in an orderly manner; FIG. 3C was a schematic diagram of application scenario 2, which generated simulated data where the activity of the gene co-expression module had a significant difference; and FIG. 3D was a schematic diagram of application scenario 3, which generated simulated data where the activity of the gene co-expression module had cell type-specificity.

Application scenario 1 aimed to generate simulated data where the activity of co-expression modules of the same gene decreased in an orderly manner. Specifically: 4 data sets containing different cell numbers were generated through SERGIO simulation, and the corresponding cell population numbers were 1,000, 3,000, 5,000, and 10,000, respectively; for each data set, 4 cell groups (A, B, C, and D) were simulated, and each cell group had 100 genes. These genes corresponded to 4 gene modules (M1, M2, M3, and M4), including 3 similarly-active gene co-expression modules (gene module M2, gene module M3, and gene module M4) and 1 gene co-expression module (gene module M1) with gradually decreasing activity in the 4 cell populations. Assuming that the target gene of the drug was located in the gene module M1, the actual drug response sequence of the 4 cell populations should be in a descending order, A>B>C>D.

Application scenario 2 aimed to generate simulated data with significant differences in the activity of a same co-expression module. Specifically: 4 data sets containing different cell numbers were generated through SERGIO simulation, and the corresponding cell population numbers were 1,000, 3,000, 5,000, and 10,000, respectively; for each data set, 6 cell groups (A, B, C, D, E, and F) were simulated, and each cell group had 100 genes. These genes corresponded to 4 gene modules, including 3 similarly-active gene co-expression modules (gene module 2, gene module 3, and gene module 4) and one gene co-expression module (gene module 1) with significantly different activity in 6 cell populations. Assuming that the target gene of the drug was located in the gene module 1, the true drug response sequence of the 6 cell populations should be that the rankings of A, B, and C were significantly higher than that of D, E, and F, that is, (A, B, C)>(D, E, F).

Application scenario 3 aimed to generate simulated data where the activities of 4 gene co-expression modules decreased in an orderly manner. Specifically: 4 data sets containing different cell numbers were generated through SERGIO simulation, and the corresponding cell population numbers were 1,000, 3,000, 5,000, and 10,000, respectively; for each data set, 4 cell groups (A, B, C, and D) were simulated, and each cell group had 100 genes. These genes corresponded to 4 gene co-expression modules, where the activity of gene module 1 in A was significantly higher than that of the other 3 gene modules; the activity of gene module 2 in B was significantly higher than that of the other 3 gene modules; the activity of gene module 3 in C was significantly higher than that of the other three gene modules; and the activity of gene module 4 in D was significantly higher than that of the other 3 gene modules. Assuming that the target genes of the drug were located in gene module 1 in A, gene module 2 in B, gene module 3 in C, and gene module 4 in D, the real drug response sequence should be A, B, C, and D in sequence.

### (2) Testing of Simulated Data:

For disease samples driven by single or multiple disease-causing genes that interacted with other genes, these genes generally formed a gene co-expression module. To this end, three different types of simulated data and three application scenarios were designed to test the prediction performance of scRank.

The results of application scenario 1 showed that the scRank could accurately sort the 4 cell populations, with an order consistent with the real situation; and as the number of cells increased, the highest ranking percentage of A cell population was greater than the percentage of other cell populations.

The results of application scenario 2 showed that the scRank could distinguish the “high group” and “low group” in gene module 1, and its ranking difference was consistent with the real situation; and as the number of cells increased, the overall ranking of A, B, and C was higher than that of D, E, and F, the more significant the difference was.

The results of application scenario 3 showed that for the perturbation of specific module genes, the scores of the drug-responsive cell populations predicted by scRank were greater than those of any other cell type; and the average scRank ranking of the target cell population was first under most conditions. The above results all proved that the scRank could accurately sort the drug-responsive cell population.

### Test Case I: Mouse Medulloblastoma

The scRNA-seq data samples of the mouse medulloblastoma and their treatment with vismodegib were from a GEO platform, with a registration number of GSE129730. Matrix data were standardized according to a workflow provided by the author (github.com/ben-babcock/Gershon_single-cell). There were 10 cell types in total, including tumor cells Node A, Node B, Node C, and Node D, neurons, astrocytes, microglial cells, endothelial cells, fibroblasts, and oligodendrocytes. This document confirmed that a target of vismodegib was Smo, and Node_B showed sensitivity to vismodegib, while Node_A showed resistance to vismodegib.

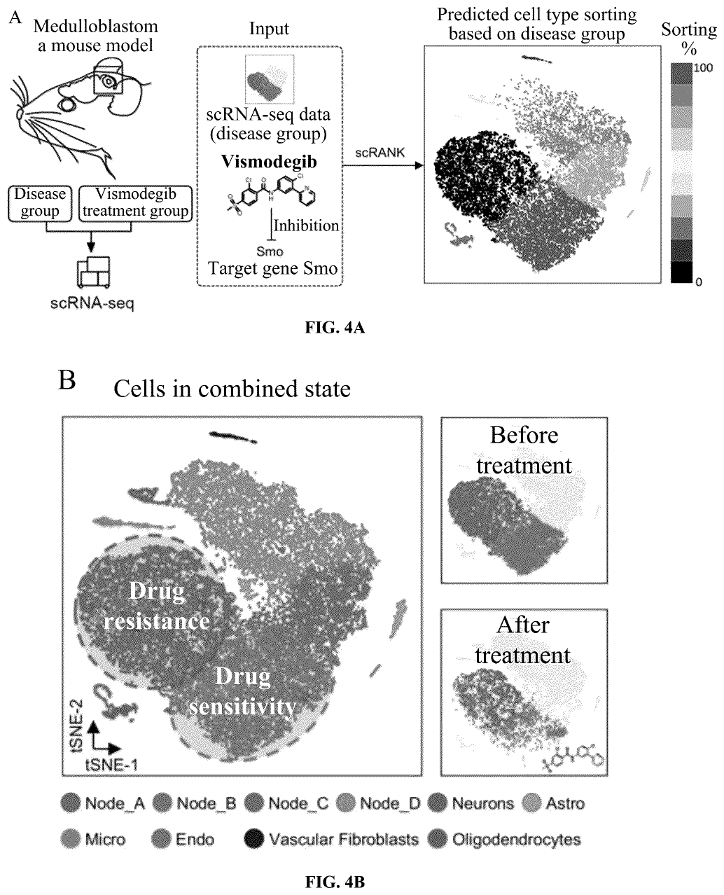

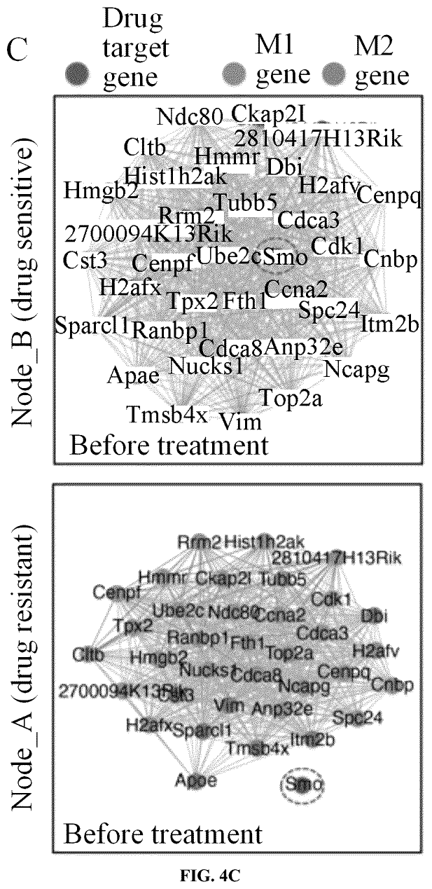

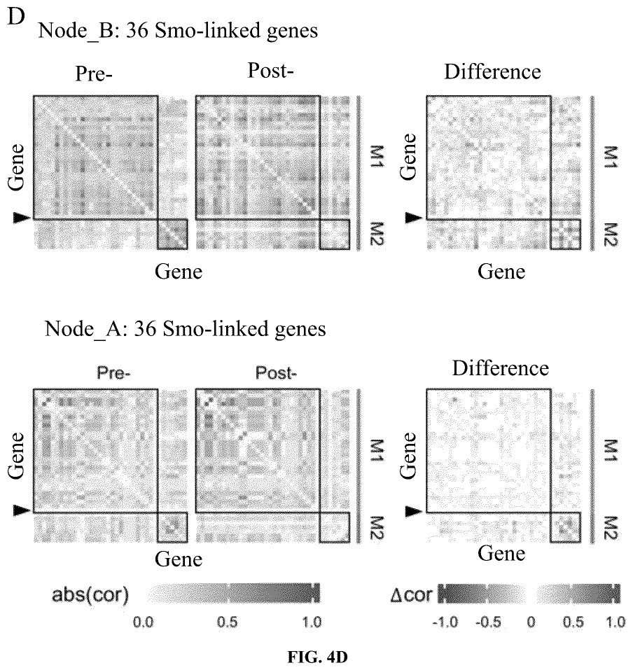

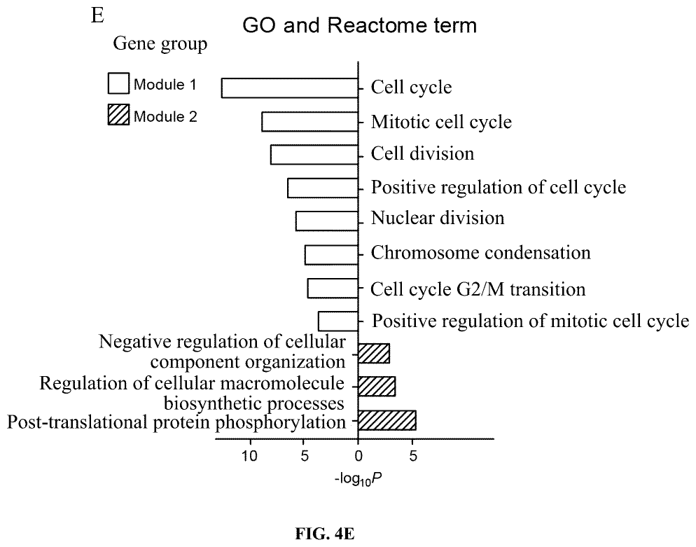

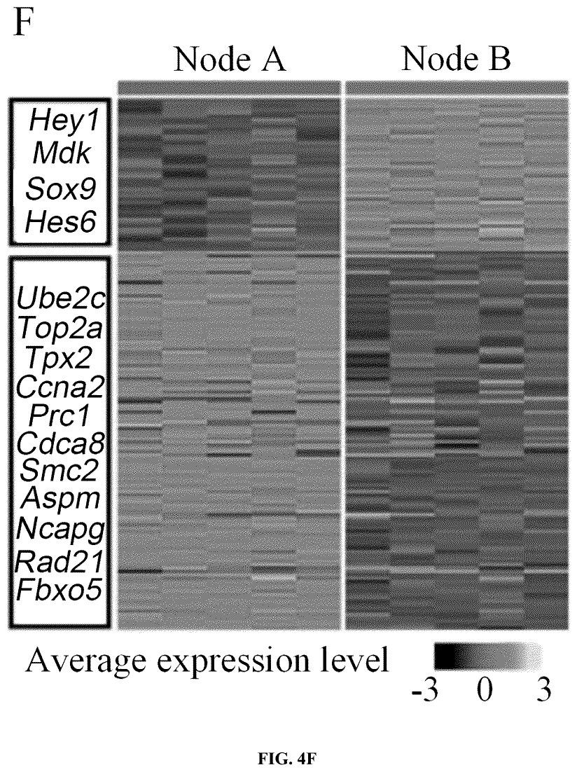

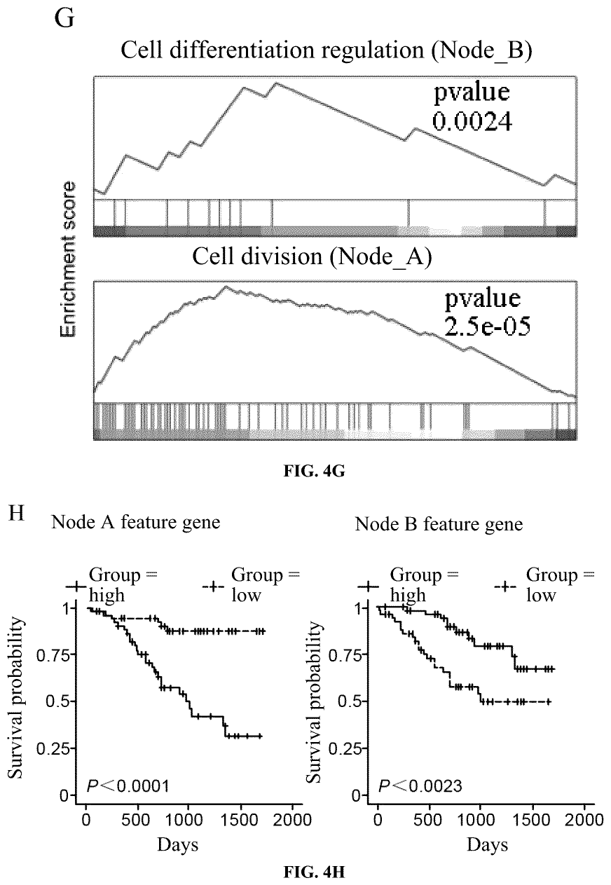

The results were shown in FIGS. 4A-H. FIGS. 4A-H show schematic diagrams for use of the scRank in mouse medulloblastoma data; where FIG. 4A was a schematic diagram of a prediction result of the scRank on mouse medulloblastoma, and target gene Smo of vismodegib and the scRNA-seq data of mouse medulloblastoma in disease states were the input of scRank; FIG. 4B was a schematic diagram of cell populations confirmed to be sensitive and resistant to vismodegib in the literature, namely Node_B and Node_A; FIG. 4C was a schematic diagram of a GRN of Node_A and Node_B under disease conditions; FIG. 4D was a schematic diagram of GRNs in two tumor subtypes and their differences before and after administration, and the value in hot soil was the weight of the edge in GRN; FIG. 4E was a schematic diagram of a GO enrichment result of gene modules M1 and M2; FIG. 4F was a schematic diagram of DEGs in the Node_A and Node_B under disease conditions; FIG. 4G was a schematic diagram of a significantly activated pathway in the Node_B and Node_A; and FIG. 4H was a prognostic analysis chart of medulloblastoma patients using feature genes of the Node_A and Node_B as classification criteria.

The scRank predicted that Node_B was the most drug-responsive cell population to vismodegib, while Node_A was the least drug-responsive cell population, which were completely consistent with the real results. The study found that Smo was a highly co-expressed gene in the GRN of Node_B under disease state, while Smo was an isolated node in the GRN of Node_A. This explained to a certain extent why Node_B was sensitive to vismodegib, while Node_A was resistant to vismodegib. Cluster analysis was conducted on the gene co-expression matrix of Node_B and Node_A, and it was found that the difference between Node_B before and after administration was significantly higher than that of Node_A, while the gene co-expression module M1 where Smo was located was also significantly enriched in signaling pathways related to the cell cycle. The differential genes of Node_A and Node_B in disease states were further compared through gene set enrichment analysis (GSEA). The results found that Node_B significantly overexpressed genes such as Hey1, Mdk, Sox9, and Hes6, and was significantly positively correlated with regulation of cell differentiation; while Node_A significantly overexpressed genes such as Ube2c, Top2a, Tpx2, and Ccna2, and was significantly positively correlated with cell division.

The above analyzes all showed that Node_A was more malignant than Node_B. Accordingly, medulloblastoma bulk sequencing data were collected from additional 178 patients. According to the feature genes of Node_A and Node_B, the 178 patients were divided into two groups: a first group was patients with high expression of Node_A and low expression of Node_A; a second group was patients with high expression of Node_B and low expression of Node_B. Combining the patient's prognosis data, it was found that compared with patients with low Node_B expression, patients with high Node_B expression had a better prognosis; while compared with patients with low Node_A expression, patients with high Node_A expression had a worse prognosis. This further confirmed that Node_A was more malignant than Node_B and verified the accuracy of scRank in predicting drug-sensitive and drug-resistant cell populations.

### Test Case II: Major Depressive Disorder (MDD)

The scRNA-seq data samples of the dorsolateral prefrontal cortex of MDD patients and their treatment with the selective serotonin reuptake inhibitor fluoxetine were from a GEO platform, with a registration number of GSE144136. This data set had a total of 25 cell types, including: excitatory neurons (Ex_1, Ex_2, Ex_3, Ex_4, Ex_5, Ex_6, Ex_7, Ex_8, Ex_9, and Ex_10), inhibitory neurons (Inhib_1, Inhib_2, Inhib_3, Inhib_5, Inhib_6, Inhib_7, and Inhib_8), endothelial cells, microglial cells, oligodendrocyte precursor cells (OPC_1, OPC_2), astrocytes (Astro_2, Astro_3), and oligodendrocytes (Oligos_1, Oligos_3). The literature confirmed that fluoxetine was a commonly used drug for treating MDD. This drug mainly inhibited SLC6A4 of excitatory neurons, thereby inhibiting the serotonin transporter, blocking serotonin reuptake, increasing the availability of serotonin, and treating the MDD.

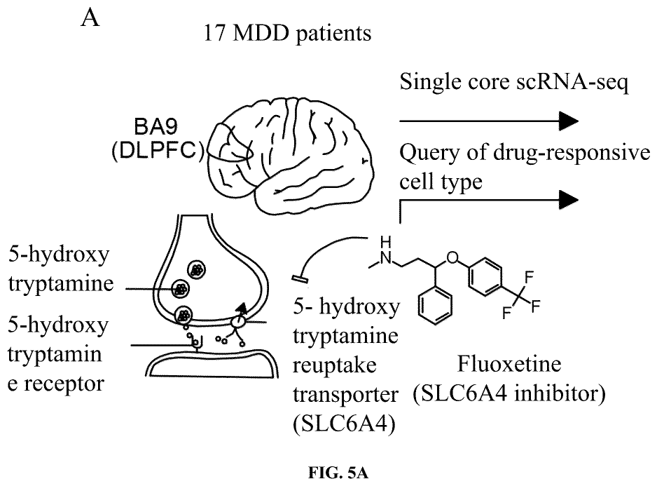

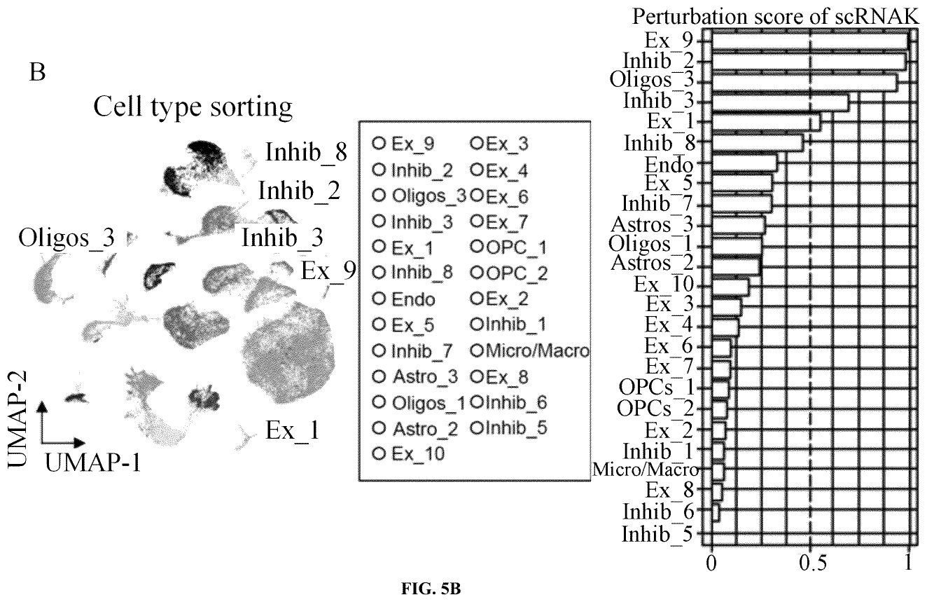

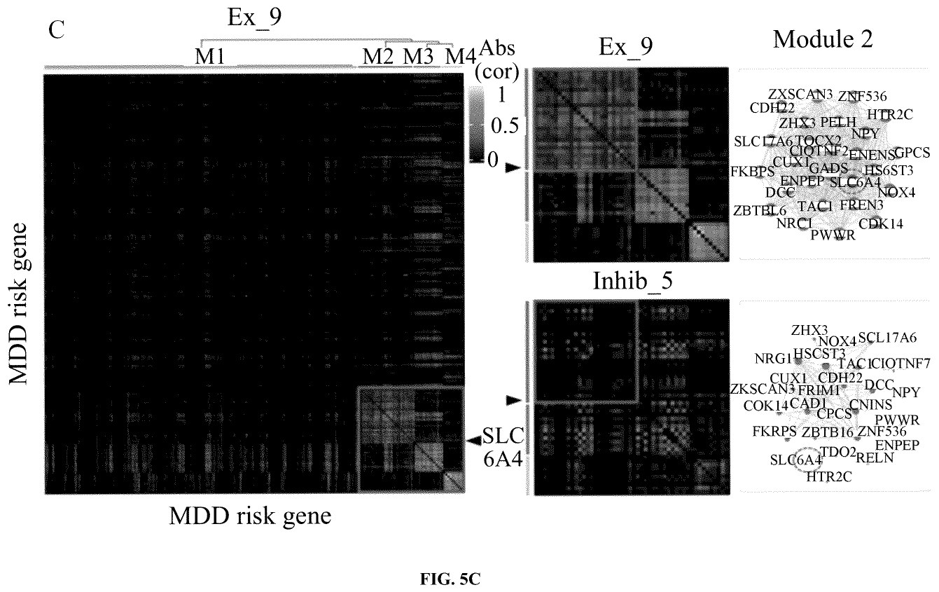

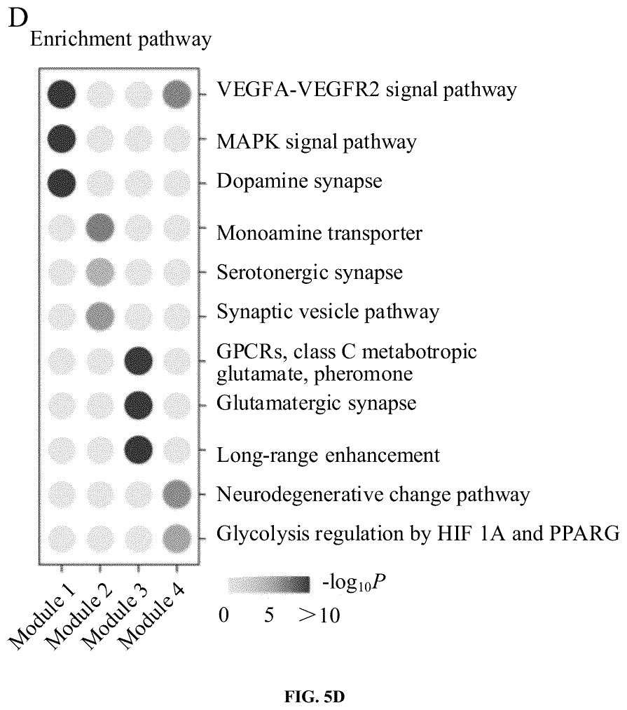

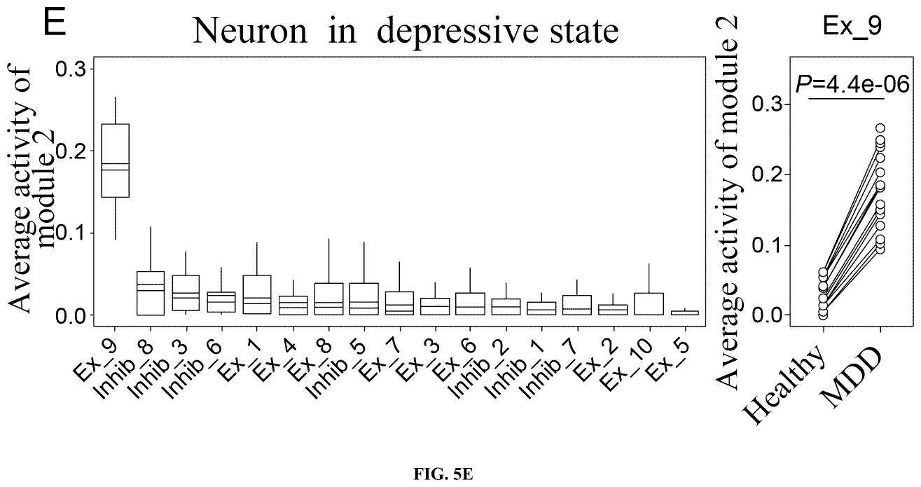

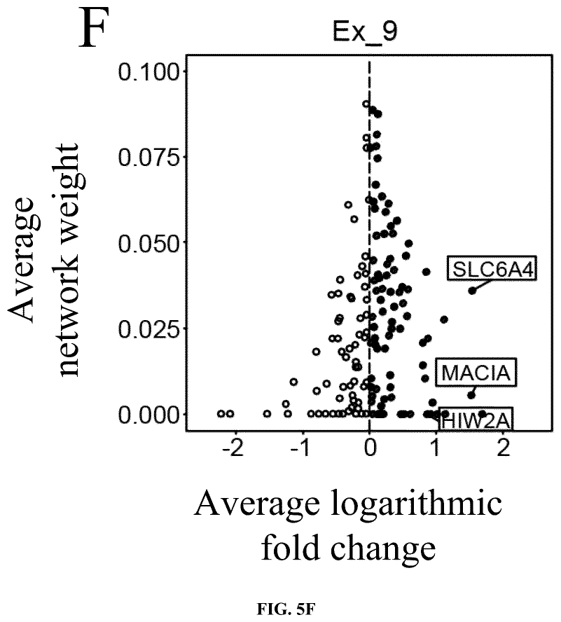

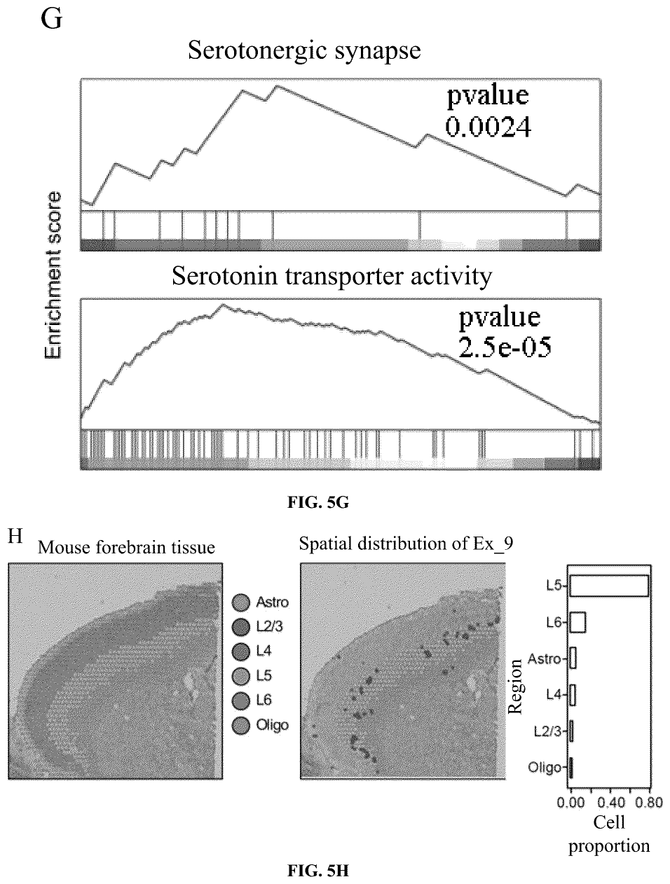

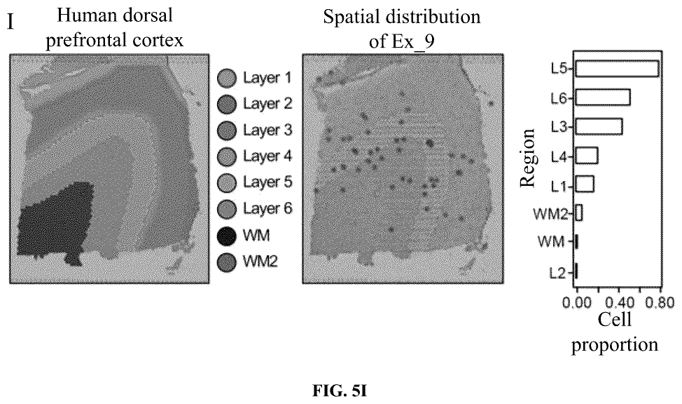

The results were shown in FIGS. 5A-I. FIGS. 5A-I showed a schematic diagram for use of the scRank in dorsolateral prefrontal cortex data of patients with major depressive disorder (MDD); where FIG. 5A was a schematic diagram of the scRNA-seq data of patients with MDD and a target SLC6A4 of a therapeutic drug fluoxetine; FIG. 5B was a schematic diagram of ranking results of all cell populations by the scRank on a data set of the patients with MDD; FIG. 5C was a schematic diagram of the GRN and cluster analysis results of Ex_9 and Inhib_5 in disease states, including 4 gene co-expression modules; FIG. 5D was a schematic diagram of pathway enrichment results of gene co-expression modules M1, M2, M3, and M4; FIG. 5E was a schematic diagram of activity distribution of the gene co-expression module M2 for all cell populations in MDD; FIG. 5F was a schematic diagram of differential genes of Ex_9 in normal person and MDD; FIG. 5G was a schematic diagram of pathways significantly enriched in Ex_9, and the vertical axis represented the GSEA pathway score; FIG. 5H was spatial distribution of different cell populations in MDD on a mouse forebrain; and FIG. 5I was spatial distribution of different cell populations in MDD on a human dorsolateral prefrontal cortex.

The scRank predicted that excitatory neuron Ex_9 was the cell population with the highest response to fluoxetine, while Inhib_5 was the least responsive cell population. To further explain this result, cluster analysis was conducted on the gene co-expression matrices of Ex_9 and Inhib_5 in disease states. The results showed that the genes used to construct GRN could be divided into 4 co-expression patterns, and the co-expression module M2 where fluoxetine's target SLC6A4 was located happened to be the most active module in Ex_9. The enrichment results also confirmed that the co-expressed genes in M2 were significantly related to neuronal synapse-related pathways, including Monoamine Transport, Serotonergic synapse, and Synaptic Vesicle Pathway, which was consistent with a mechanism of action in fluoxetine. The above results indicated that the gene co-expression module M2 where SLC6A4 was located played a crucial role in the treatment of MDD with fluoxetine. Further focusing on the M2 gene co-expression module, it was found that the M2 activity of Ex_9 was much higher than that of other cell types, and the M2 activity of Ex_9 was also significantly higher than that of the normal group. Meanwhile, the highly expressed gene of Ex_9 in disease states was also significantly related to synapses. These results indicated that the highly expressed M2 gene co-expression module in Ex_9 was highly related to both the pathogenesis of MDD and the mechanism of action of fluoxetine in the treatment of MDD.

Research showed that fluoxetine mainly acted on deep neurons. To this end, cell populations in MDD were mapped to spatial transcriptome maps (10×Visium datasets) of mouse forebrain and human dorsolateral prefrontal cortex using a CellTrek tool. The results showed that Ex_9 targeted by fluoxetine was indeed mainly distributed on neurons in the deep layers of brain tissue, with most of the Ex_9 cells mainly concentrated in the L5 and L6 layers. The above results further confirmed that the cell population targeted by fluoxetine when exerting its therapeutic effect was Ex_9. By inhibiting SLC6A4 of Ex_9, serotonin reuptake was blocked, thereby treating the MDD.

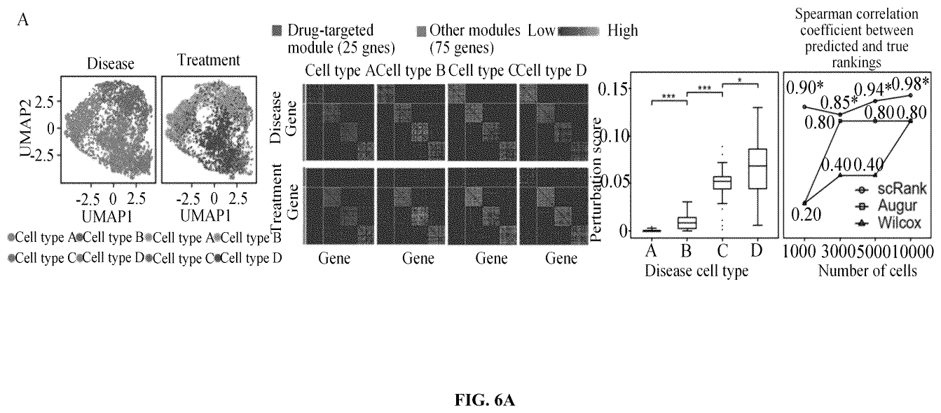

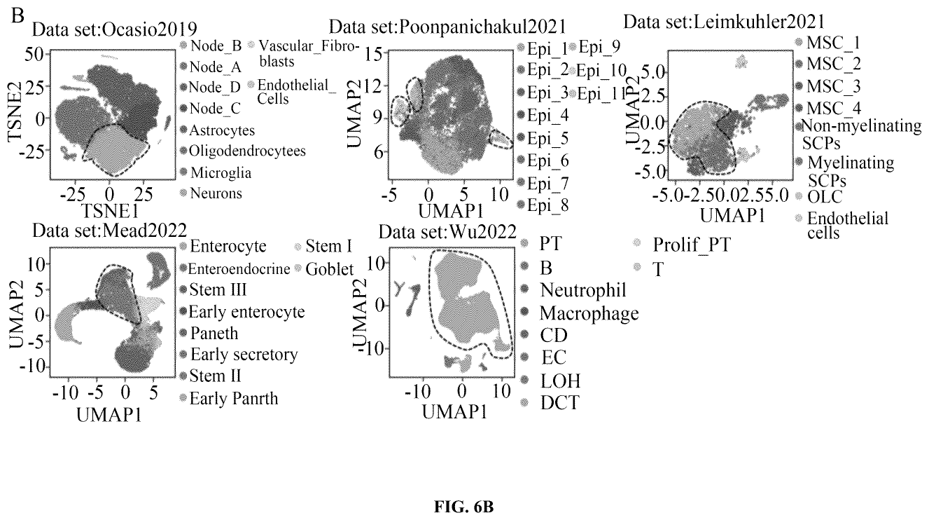

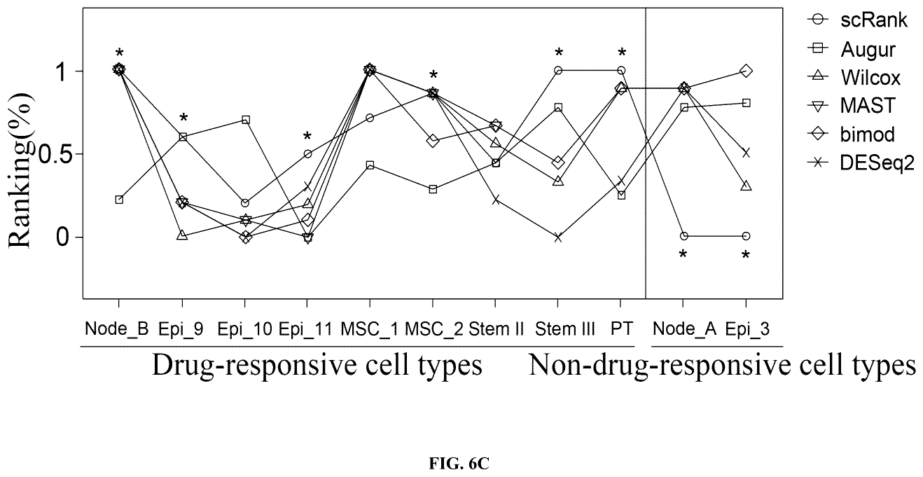

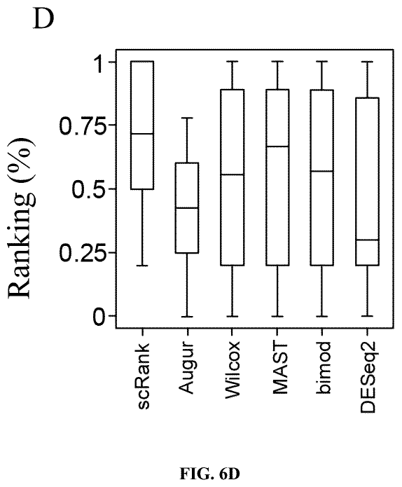

5 sets of real drug-perturbed scRNA-seq data sets were collected from public databases through literature search. Each dataset contained matrix data for two states, disease and drug administration, with clear positive results for response to the drug and negative results for no response to the drug. Therefore, scRank was benchmarked using these five sets of real data as shown in FIGS. 6A-D, and its performance was compared with that of existing commonly used strategies and methods. FIGS. 6A-D showed a comparison of the scRank with other methods; where FIG. 6A was a schematic diagram comparing the performances of scRank, Augur, and Wilcox with simulated data; FIG. 6B was a schematic diagram of five sets of real drug-perturbed scRNA-seq data sets; FIG. 6C was a schematic diagram comparing the performances of scRank, Augur, Wilcox, MAST, bimod, and DESeq2 with simulated data; and FIG. 6D was box distribution of the prediction results with different methods.

### (1) Collection of Real Data

Five real-life drug-perturbed scRNA-seq data sets included mouse medulloblastoma, human colorectal cancer epithelial cells, mouse fibrotic bone marrow, intestinal miniaturized organoids, and mouse diabetic nephropathy datasets. AC specific collection process was as follows:

The scRNA-seq data samples of the mouse medulloblastoma and their treatment with vismodegib were from a GEO platform, with a registration number of GSE129730. Matrix data were standardized according to a workflow provided by the author (github.com/ben-babcock/Gershon_single-cell). There were 10 cell types in total, including tumor cells Node_A, Node B, Node C, and Node D, neurons, astrocytes, microglial cells, endothelial cells, fibroblasts, and oligodendrocytes.

The scRNA-seq data samples of the human colorectal cancer epithelial cells and their treatment with leucovorin and 5-fluorouracil administration were from a GEO platform, with a registration number GSE155953. There were 11 different types of epithelial cells, namely Epithelial cell 1, Epithelial cell 2, Epithelial cell 3, Epithelial cell 4, Epithelial cell 5, Epithelial cell 6, Epithelial cell 7, Epithelial cell 8, Epithelial cell 9, Epithelial cell 10, and Epithelial cell 11.

The scRNA-seq data samples of mouse fibrotic bone marrow and tasquinimod administration were from a GEO platform, with a registration number of GSE156644. There were a total of 8 cell types, namely mesenchymal stromal cells (MSC_1, MSC_2, MSC_3, and MSC_4), non-myelinating Schwann cell precursors (SCPs), myelinating Schwann cell precursors (myelinating SCP), steoblastic lineage cells (OLCs), and endothelial cells.

Intestinal miniaturized organoids and their scRNA-seq data after KPT-330 treatment were from a Single-Cell Portal platform (singlecell.broadinstitute.org/single_cell, SCP1547). The data set included 10 cell types, namely stem cells (I, II, III), enterocytes, early enterocytes, enteroendocrine cells, Paneth cells, early Paneth cells, early secretory cells, and goblet cells.

The scRNA-seq data samples of the mouse diabetic nephropathy and their treatment with dapagliflozin were from a GEO platform, with a registration number of GSE181382. There were 10 cell types in total, including proximal tubule cells, B cells, neutrophils, macrophages, collecting duct cells, endothelial cells, ascending loop of Henle, distal convoluted tubule cells, proliferating proximal tubule cells, and T cells.

(2) Real Data Test of scRank

The prediction performance of scRank was tested on five sets of real data, including data sets from mouse medulloblastoma, human colorectal cancer epithelial cells, mouse fibrotic bone marrow, intestinal miniaturized organoids, and mouse diabetic nephropathy:

A first set of data was scRNA-seq data of the mouse medulloblastoma and their treatment with vismodegib, where the target of vismodegib was Smo. This document confirmed that Node_B was sensitive to vismodegib, while Node_A was resistant to vismodegib. The results showed that the scRank predicted that Node_B was the most drug-responsive cell population to vismodegib, while Node_A was the least drug-responsive cell population, which were completely consistent with the real results.

A second set of data was scRNA-seq data of the human colorectal cancer epithelial cells and their administration with leucovorin and 5-fluorouracil, where the targets of leucovorin and 5-fluorouracil were TOP1 and TYMS, respectively. This document confirmed that Epithelial cell 9, Epithelial cell 10, and Epithelial cell 11 were the most sensitive to the combined administration of leucovorin and 5-fluorouracil, while Epithelial cell 3 showed resistance. The results showed that scRank predicted that Epithelial cell 9, Epithelial cell 10, and Epithelial cell 11 would rank in the top 50%, while Epithelial cell 3 was the least drug-responsive cell population, which were basically consistent with the real results.

A third set of data was scRNA-seq data of the mouse fibrotic bone marrow and their treatment with tasquinimod, where the targets of tasquinimod were S100a8 and S100a9. This document confirmed that the target cell populations on which tasquinimod acted were MSC_1 and MSC_2. The results showed that the scRank predicted that the drug response of MSC_1 and MSC_2 ranked in the top 25%, which were basically consistent with the real results.

A fourth set of data was scRNA-seq data of the intestinal miniaturized organoids and their treatment with KPT-330, where the target of KPT-330 was Xpo1. This document confirmed that the target cell populations on which KPT-330 acted were stem cells II and III. The results showed that the scRank predicted that the drug response ranking of stem cell III was top1, which was completely consistent with the real results.

A fifth set of data was scRNA-seq data of the mouse diabetic nephropathy and their treatment with dapagliflozin, where the target of dapagliflozin was Slc5a2. This document confirmed that the target cell population acted by dapagliflozin was proximal tubule cells (PTs). The results showed that the scRank predicted that the drug response ranking of PTs was top1, which was completely consistent with the real results.

(3) Real Data Comparison Between scRank and Other Methods

Performance tests were conducted on simulated data and real data separately. Considering that other methods required two states of scRNA-seq data, the gene expression profile matrix of a specific cell population was automatically generated with the Python package of “SERGIO” and the predefined GRN module. Specifically: 4 data sets containing different cell numbers were generated through SERGIO simulation, and the corresponding cell population numbers were 1000, 3000, 5000, and 10000, respectively; for each data set, 4 cell groups (A, B, C, and D) were simulated, and each cell group had 100 genes. These genes corresponded to 4 gene modules, including 3 similarly-active gene co-expression modules (gene modules M2, M3, and M4) and 1 gene co-expression module (gene module M1) with gradually decreasing activity in the 4 cell populations, which served as scRNA-seq data in disease states. For each data set, 4 corresponding cell groups (A′, B′, C′, and D′) were simulated, and each cell group had 100 genes. These genes corresponded to 4 gene co-expression modules, including 3 similarly-active gene co-expression modules (M2, M3, and M4) and 1 gene co-expression module (M1) with activity equal to M1 in A. If it was assumed that the target gene of the drug was located in the gene module M1, the actual drug response sequence of the 4 cell populations should be in a descending order after gene knockout, A>B>C>D. As shown in FIG. 6A, the scRank could basically accurately predict the real drug response ranking, with a correlation coefficient of not less than 0.9.

The most commonly used strategy at present was to determine the cell population that responded to a drug based on the number of differential genes before and after drug administration. Therefore, the scRank was compared with the four most commonly used methods for screening differential genes, including Wilcox, MAST, bimod, and DESeq2. As shown in FIG. 6B to FIG. 6D, Augur, Wilcox, MAST, bimod, and DESeq2 were generally able to accurately predict most of the cell populations that responded to drugs, and the prediction results of scRank were significantly better than those of these methods. It is worth noting that other methods could not accurately predict insensitive negative results. It was seen that the scRank could not only predict the drug-responsive cell populations, but also predict the drug-non-responsive cell populations.

### Example 3

To implement the method corresponding to Example 1 to achieve corresponding functions and technical effects, a system for sorting a drug-responsive cell population based on scRNA-seq data was provided below.

The present disclosure provided a system for sorting a drug-responsive cell population based on scRNA-seq data, including:
a data and information acquisition module configured to acquire scRNA-seq data and drug target information of cell populations in a disease state;
a GRN construction module configured to construct a target gene regulatory network (GRN) for each of the cell populations based on a co-expression relationship of genes and the scRNA-seq data;
a tpGRN construction module configured to virtually knock out targets in the GRN according to the drug target information, and construct a target-perturbed gene regulatory network (tpGRN) for each of the cell populations;
a manifold alignment configured to acquire a low-dimensional representation of each of network nodes from the GRN in the GRN and the tpGRN through manifold alignment; where the network nodes are gene nodes;
a Euclidean distance calculation module configured to calculate a Euclidean distance for each of the network nodes between the GRN and the tpGRN through a Euclidean distance based on the low-dimensional representation;
a drug perturbation score generation module configured to score a drug response of each of the cell populations based on the distance by comprehensively considering changing trends of a drug target, a 2-hop node, and an edge of the 2-hop node in the GRN and the tpGRN, and generate a drug perturbation score; and
a sorting module configured to sort the drug response of each of the cell populations according to the drug perturbation score, and determine a sorting result of the drug-responsive cell population; where the sorting result of the drug-responsive cell population is used to characterize a degree of response of each of the cell populations to drug perturbation.

Each embodiment in the description is described in a progressive mode, each embodiment focuses on differences from other embodiments, and references may be made to each other for the same and similar parts between embodiments. Since the system disclosed in an embodiment corresponds to the method disclosed in an embodiment, the description is relatively simple, and for related contents, references may be made to the description of the method.

Particular examples are used herein for illustration of principles and implementation modes of the present disclosure. The descriptions of the above embodiments are merely used for assisting in understanding the method of the present disclosure and its core ideas. In addition, those of ordinary skill in the art may make various modifications in terms of particular implementation modes and the scope of application in accordance with the ideas of the present disclosure. In conclusion, the content of the description shall not be construed as limitations to the present disclosure

Various embodiments of the disclosure may have one or more of the following effects. In some embodiments, the disclosure may provide a method for sorting a drug-responsive cell population based on single-cell RNA sequencing (scRNA-seq) data, to avoid a low accuracy in inferring the drug-responsive cell population. In other embodiments, the present disclosure may provide a method and a system for sorting a drug-responsive cell population based on scRNA-seq data. In an example of the method, a target gene regulatory network (GRN) may be constructed for each of the cell populations using only scRNA-seq data in a disease state, targets in the GRN may be virtually knocked out using drug target information, and a target-perturbed gene regulatory network tpGRN may be constructed for each of the cell populations, a drug response of each of the cell populations may be scored based on the GRN and the tpGRN, and the drug response of each of the cell populations may be sorted according to a drug perturbation score, and a sorting result of the drug-responsive cell population may be determined. In further embodiments, an accuracy of inference on the drug-responsive cell population may be improved based on prior knowledge of the drug targets. Moreover, the drug responses of the cell populations may be obtained using only the scRNA-seq data in a disease state, thereby expanding an application scope of only the scRNA-seq data in a disease state. In some embodiments, the present disclosure may provide a method and a system for sorting a drug-responsive cell population based on scRNA-seq data, to improve an accuracy in inferring the drug-responsive cell population

Many different arrangements of the various components depicted, as well as components not shown, are possible without departing from the spirit and scope of the present disclosure. Embodiments of the present disclosure have been described with the intent to be illustrative rather than restrictive. Alternative embodiments will become apparent to those skilled in the art that do not depart from its scope. A skilled artisan may develop alternative means of implementing the aforementioned improvements without departing from the scope of the present disclosure.

It will be understood that certain features and subcombinations are of utility and may be employed without reference to other features and subcombinations and are contemplated within the scope of the claims. Unless indicated otherwise, not all steps listed in the various figures need be carried out in the specific order described.

The disclosure claimed is:

1. A method for sorting a drug-responsive cell population based on single-cell RNA sequencing (scRNA-seq) data, comprising:
acquiring scRNA-seq data and drug target information of cell populations in a disease state;
constructing a target gene regulatory network (GRN) for each of the cell populations based on a co-expression relationship of genes and the scRNA-seq data;
virtually knocking out targets in the GRN according to the drug target information, and constructing a target-perturbed gene regulatory network (tpGRN) for each of the cell populations;
acquiring a low-dimensional representation of each of network nodes from the GRN in the GRN and the tpGRN through manifold alignment; wherein the network nodes are gene nodes;
calculating a Euclidean distance for each of the network nodes between the GRN and the tpGRN through a Euclidean distance based on the low-dimensional representation;
scoring a drug response of each of the cell populations based on a distance by considering changing trends of a drug target, a 2-hop node, and an edge of the 2-hop node in the GRN and the tpGRN, and generating a drug perturbation score; and
sorting the drug response of each of the cell populations according to the drug perturbation score, and determining a sorting result of the drug-responsive cell population; wherein the sorting result of the drug-responsive cell population is used to characterize a degree of response of each of the cell populations to drug perturbation.

2. The method according to claim 1, wherein a process of constructing the GRN for each of the cell populations based on the co-expression relationship of the genes and the scRNA-seq data comprises:
constructing a scRNA-seq expression matrix according to the scRNA-seq data;
randomly selecting δc cells from each of cell types in the scRNA-seq expression matrix, extracting a gene expression matrix corresponding to each of the selected δc cells, and repeating S times; wherein δ represents a selection ratio and c represents a number of cells;
subjecting genes in the gene expression matrix to feature selection to construct a gene set of the GRN; wherein the gene set comprises top 2,000 highly variable genes in the scRNA-seq data, genes corresponding to transcription factors in an AnimalTFDB database, and target genes of drugs in a DGIdb database;
determining a low-dimensional representation of each of the genes in the gene set by principal component analysis and estimating a regression coefficient between the genes as a weight of an edge in the GRN;
defining any one of gene expression matrices, subjecting a target gene on multiple potential covariations using the principal component analysis, and constructing a relationship between the target gene and a regulatory gene;
constructing a gene adjacency matrix according to the relationship between the target gene and the regulatory gene as well as the weight;
combining multiple gene adjacency matrices into a third-order tensor χ of S×T×R using tensor component analysis; wherein S represents a number of the GRNs, T represents a number of the target genes, and R represents a number of the regulatory genes;
extracting a main feature pattern shared by a plurality of the GRNs, decomposing and approximating the third-order tensor χ to generate a new third-order tensor; and
conducting normalization on a result obtained through dividing each of the weights by a maximum absolute value in all weight absolute values based on the new third-order tensor, and constructing a final adjacency matrix for each of the cell populations; wherein the final adjacency matrix is the GRN.

3. The method according to claim 2, wherein the relationship between the target gene and the regulatory gene is:
$$Y = {X}^{′} {β}^{′} + ε ,$$
wherein:
Y represents an expression vector of the target gene;
X′ represents expression vectors of the regulatory genes corresponding to multiple top-ranked principal components;
β′ represents a regression coefficient of a transformed potential covariate; and
ε represents a random error.

4. The method according to claim 2, wherein a process of extracting the main feature pattern shared by the multiple GRNs, decomposing and approximating the third-order tensor χ to generate the new third-order tensor comprises:
extracting the main feature pattern shared by the multiple GRNs, and decomposing and approximating the third-order tensor χ with a formula χ≈Σᵣ₌₁^{R} s^{r}⊗t^{r}⊗r^{r}; wherein s^{r} represents a vector of a factor r in a GRN number dimension; ⊗ represents an outer product operation; t^{r} represents a vector of the factor r in a target gene dimension; and r^{r} represents a vector of the factor r in a regulatory gene dimension; and
selecting first five factors to characterize key components of the third-order tensor χ, and generating a new third-order tensor through cumulative averaging of the key components.

5. The method according to claim 2, wherein a process of virtually knocking out the targets in the GRN according to the drug target information, and constructing the tpGRN for each of the cell populations comprises:
extracting a row where a drug target gene is located from the final adjacency matrix, and setting a corresponding value of the row where the drug target gene is located to 0, to virtually construct a drug-perturbed adjacency matrix; wherein the drug-perturbed adjacency matrix is the tpGRN.

6. The method according to claim 5, wherein a process of acquiring the low-dimensional representation of each of the network nodes from the GRN in the GRN and the tpGRN through manifold alignment comprises:
combining the final adjacency matrix and the drug-perturbed adjacency matrix into a combined matrix; and
projecting the combined matrix to a shared low-dimensional potential space with the manifold alignment, determining the low-dimensional representation of each of the gene nodes in the GRN and the tpGRN, and interpreting the Euclidean distance between each of matched gene nodes as a perturbation effect of a drug on the cell types.

7. The method according to claim 6, wherein the combined matrix is:
$$w = [ \begin{matrix}{A}^{′} & λ I \\ λ I & {A}_{PB}^{′}\end{matrix} ] ,$$
wherein:
W represents the combined matrix;
A′ represents the final adjacency matrix;
A_{PB}′ represents the drug-perturbed adjacency matrix;
λ represents an adjustment parameter; and
I represents an identity matrix describing a correspondence between the gene nodes in the GRN and the tpGRN.

8. The method according to claim 1, wherein the drug perturbation score score is:
$$score = \frac{{D}_{Tar} {W}_{o u t}}{{Deg}_{T a r}} + {\sum }_{n}^{N} {D}_{n} {W}_{i n} + {\sum }_{n}^{N} \frac{{D}_{n} {W}_{o u t}}{{Deg}_{n}} ,$$
wherein:
D_{Tar} represents the distance of the target gene between the GRN and the tpGRN;
Wₒᵤₜ represent a weight of an edge of a gene node leaving the GRN;
Deg_{Tar} is a degree of the target gene in the GRN;
N represents a total number of genes regulated by the target gene;
n represents a gene sequence number;
Dₙ represents a distance of a downstream gene between the GRN and the tpGRN;
Wᵢₙ represents a weight of an edge of a gene node entering the GRN; and
Degₙ represents a degree of the downstream genes in the GRN.

9. A system for sorting a drug-responsive cell population based on scRNA-seq data, comprising:
a data and information acquisition module configured to acquire scRNA-seq data and drug target information of cell populations in a disease state;
a GRN construction module configured to construct a target gene regulatory network (GRN) for each of the cell populations based on a co-expression relationship of genes and the scRNA-seq data;
a tpGRN construction module configured to virtually knock out targets in the GRN according to the drug target information, and construct a target-perturbed gene regulatory network (tpGRN) for each of the cell populations;
a manifold alignment configured to acquire a low-dimensional representation of each of network nodes from the GRN in the GRN and the tpGRN through manifold alignment; wherein the network nodes are gene nodes;
a Euclidean distance calculation module configured to calculate a Euclidean distance for each of the network nodes between the GRN and the tpGRN through a Euclidean distance based on the low-dimensional representation;
a drug perturbation score generation module configured to score a drug response of each of the cell populations based on a distance by considering changing trends of a drug target, a 2-hop node, and an edge of the 2-hop node in the GRN and the tpGRN, and generate a drug perturbation score; and
a sorting module configured to sort the drug response of each of the cell populations according to the drug perturbation score, and determine a sorting result of the drug-responsive cell population; wherein the sorting result of the drug-responsive cell population is used to characterize a degree of response of each of the cell populations to drug perturbation.
